(19) United States
(12) Patent Application Publication
(10) Pub. No.: US 2021/0353815 A1
(43) Pub. Date: Nov. 18, 2021

(54) SCENTED COLOR-CHANGING SYSTEM

(71) Applicant: AKI, Inc., Chattanooga, TN (US)

(72) Inventors: Mickael Devassine, Ooltewah, TN (US); David O'Halloran, Milltown, NJ (US); Josh M. Dozier, Lafayette, GA (US)

(73) Assignee: AKI, Inc., Chattanooga, TN (US)

(21) Appl. No.: 17/302,823

(22) Filed: May 13, 2021

Related U.S. Application Data
(60) Provisional application No. 63/024,217, filed on May 13, 2020.

(51) Int. Cl. A61L 9/12; A61L 9/04; C09B 9/00

(52) U.S. Cl. CPC A61L 9/12; C09B 9/00; A61L 9/044; A61L 9/042

(57) ABSTRACT

A scented color-changing system for revealing a hidden message, brand, picture, and the like on a substrate such as a paper when a fragrance is applied or released on the substrate. The hidden message contains a color former such as a leuco dye and the fragrance contains a color developer such as zinc chloride that reacts with the color former to reveal the message. The fragrance can be sprayed on the substrate or released from microcapsules embedded in or disposed on the substrate by rupturing the microcapsules. The scented color-changing system is particularly suited for use with fragrance test strips, advertising mailers, magazine inserts, and the like.

### CROSS REFERENCE TO RELATED APPLICATIONS

This application claims priority to U.S. Provisional Application Ser. No. 63/024,217, filed May 13, 2020, which is incorporated herein by reference in its entirety.

### FIELD OF THE APPLICATION

The disclosed subject matter generally relates to a color-changing system, and more particularly to a scented color-changing system for changing colors on a substrate with the application or release of a fragrance.

### DESCRIPTION OF THE RELATED ART

Color-changing compounds such as leuco dyes are commonly used in a wide variety of products from litmus papers/pH tests to novelty “disappearing ink” and many others. In such cases, a change in conditions, such as a chemical reaction, causes the compounds to change colors. There are known color-changing systems. For example, U.S. Pat. No. 5,485,792 describes a color-changing system that produces a latent color image by reacting a color developer contained in a marker with a color former applied on a support. And U.S. Pat. No. 6,124,377 describes a marking system that minimizes messiness comprising a marking instrument loaded in color former and a substrate coated with at least one color developer.

The fragrance industry—perfumes, colognes, after-shaves, air fresheners, home fragrances, and the like—is a popular and competitive industry. As in any market, it is desirable to create memorable impressions in the minds of consumers and potential customers. This poses specific challenges for fragrances. On one hand, human olfactory perception can form long-lasting memories, and the sense of smell is sometimes referred to as the “memory sense.” M. Hopkin, Link proved between senses and memory, Nature, doi:10.1038/news040524-12, 2004. On the other hand, the sense of smell is “notoriously unreliable, but shows substantial benefits from visual cues.” J. Gottfried & R. Dolan, The Nose Smells What the Eye Sees: Crossmodal Visual Facilitation of Human Olfactory Perception, Neuron, vol. 39 issue 2, 2003. Therefore, there is need for associating such visual cues with fragrances to enhance memory retention in consumers.

While there are known systems capable of changing color under certain condition changes, there remains a need for a scented color-changing system for changing colors on a substrate with the application or release of a fragrance.

### SUMMARY

The purpose and advantages of the disclosed subject matter are set forth in the following description, and additional advantages may be obtained by the practice and knowledge of those skilled in the art based on this description.

The disclosed subject matter provides, according to some embodiments, a new scented color-changing system. In some embodiments, the scented color-changing system comprises a substrate (e.g., paper) and a fragrance (e.g., perfume). The substrate has a color-changing portion having a message, brand, logo or the like written or drawn in invisible ink thereon. The ink contains a color former such as a leuco dye.

According to some embodiments, in addition to volatile fragrance molecules, the fragrance contains a dissolved color developer such as zinc chloride that can react with the color former to change the initial color of the ink to a changed color (e.g., from invisible/transparent to blue). The fragrance can be applied to the color-changing portion (and thereby the color former mixed and reacted with the color developer) by any appropriate means, such as by spraying. The fragrance can also be encapsulated in microcapsules and disposed on the substate, with an optional barrier layer between the fragrance and ink, so that the fragrance is released when the microcapsules are ruptured, such as by abrading (e.g., scratching) the substrate or peeling away a covering flap.

The foregoing summary and the following detailed description are exemplary and are intended to provide an explanation of the disclosed subject matter, which is further illustrated and explained by the accompanying drawings.

### BRIEF DESCRIPTION OF THE DRAWINGS

FIGS. 1(a)-(b) are views of an exemplary scented color-changing system in accordance with the disclosed subject matter.

FIG. 2 is a vertical cross-sectional view of substrate coatings of an exemplary scented color-changing system in accordance with the disclosed subject matter.

FIGS. 3(a)-(b) are views of an exemplary scented color-changing system in accordance with the disclosed subject matter.

FIG. 4 is a vertical cross-sectional view of substrate coatings of an exemplary scented color-changing system in accordance with the disclosed subject matter.

FIGS. 5(a)-(b) are views of an exemplary scented color-changing system in accordance with the disclosed subject matter.

FIG. 6 is a vertical cross-sectional view of substrate coatings of an exemplary scented color-changing system in accordance with the disclosed subject matter.

### DETAILED DESCRIPTION

The scented color-changing system presented herein allows a user to reveal or modify a message, brand, picture, and the like on a substrate such as a paper when a fragrance is applied or released on the substrate. The scented color-changing system is particularly suited for use with fragrance test strips, advertising mailers, magazine inserts, and the like.

In accordance with the disclosed subject matter, as embodied and broadly described herein, certain embodiments of the scented color-changing system comprise a fragrance, a substrate, and at least two reagents that generate the color-change reaction on the substrate in the presence of the fragrance. The fragrance is used as a solvent media thus allowing the chemical reaction between the reagents.

Any suitable fragrance composition containing volatile fragrance molecules can be used. For example, the fragrance can be in solution (anhydrous or water-based solution) or emulsified (oil/water or water/oil) or encapsulated. In embodiments, the fragrance is used as solvent or carrier in the color-change reaction.

Any suitable substrate can be used. In embodiments, the substrate is preferably an absorbent support such as paper. In embodiments, one or more additives such as surfactant, other solvents, antioxidants, buffers, dye inhibitors can be added to the fragrance solution or to the substrate coatings.

Any suitable reagents can be used, provided their combination and reaction generates a color change. In embodiments, one reagent, the color developer, is preferably dispersed or dissolved in an invisible ink applied on the substrate, while the other reagent, the color activator, can be encapsulated with the fragrance or dissolved or dispersed in the fragrance or in an invisible coating applied on the substrate. When the fragrance containing the color activator is applied to or release from the substrate containing the color developer (e.g., by spraying perfume on the substrate or rubbing the fragrance-infused substrate), the reagents react and generate the color change.

The color change can be generated by any suitable color former/color developer reactions, halochromic compound/acid-base reactions, or oxidation-reduction reactions. In the case of the use of a color developer and a color former (e.g., leuco dye), the color developer reacts chemically with the color former to produce a chromophore leading to a color apparition on the substrate when the fragrance is applied or released on the substrate. The terms “color former” and “color developer” are used throughout this application to refer to any such suitable color-changing reagents. The initial (i.e., pre-reaction) and changed (i.e., post-reaction) colors of the color former can be any suitable colors, including colorless, that allow an observer to observe a change. For example, in some embodiments, the initial color is colorless and the changed color is blue.

In embodiments, the color former is preferably dispersed or dissolved in a transparent ink and applied on the substrate in the form of a message, a brand, a picture, and the like by printing machine or other suitable methods.

The color developer can be dissolved in the fragrance, or it can be dispersed or dissolved in a transparent ink or coating and applied on the substrate. An intermediate coating may be applied on the substrate between the first and second reagent layers to avoid a preliminary color reaction between the reagents before the application of the fragrance. The color change reaction occurs when the fragrance is applied on the substrate (e.g., by spraying).

Alternatively, the color developer can be dissolved in the fragrance then that solution/mixture can be encapsulated. In such embodiments, the color message appears when the surface of the substrate is rubbed, thereby breaking the capsules and releasing the fragrance/color developer mixture.

Any suitable color reaction/reagents can be used in accordance with the disclosed subject matter. An example of color reaction involves the reaction between a colorless leuco dye and a color developer in presence of a fragrance. A variety of leuco dyes are suitable for the color-changing reaction and can be employed alone or in combination. These include, for example, triphenylmethane-phthalide Leuco compounds, triallylmethane Leuco compounds, fluoran Leuco compounds, phenothiazine Leuco compounds, thiofluoran Leuco compounds, xanthene Leuco compounds, indophthalyl Leuco compounds, spiropyran Leuco compounds, azaphthalide Leuco compounds, couromeno-pyrazole Leuco compounds, methine Leuco compounds, rhodamineanilino-lactam Leuco compounds, rhodaminelactam Leuco compounds, quinazoline Leuco compounds, diazaxanthene Leuco compounds and bislactone Leuco.

Commercial Leuco dye examples suitable for application in the color-changing system include but are not limited: 9′-[Ethyl(3-methylbutyl)amino]-3H-spiro[2-benzofuran-1,12′-benzo[a]xanthen]-3-one (YAMADA RED 500—CAS No. 115392-27-3), 6′-[ethyl(p-tolyl)amino]-2′-methylspiro[isobenzofuran-1(3H),9′-[9H]xanthene]-3-one (YAMADA RED 520—CAS No. 42228-32-0), crystal violet lactone (YAMADA CVL-K—CAS No. 1552-42-7), 2′-anilino-6′-[ethyl(3-methylbutyl)amino]-3′-methylspiro[isobenzofuran-1(3H),9′-[9H]xanthene]-3-one (YAMADA S-205—CAS No. 70516-41-5), 2-Anilino-6-dibutylamino-3-methylfluoran (YAMADA BLACK 400—CAS No. 89331-94-2), 2′-anilino-6′-[ethyl(p-tolyl)amino]-3′-methylspiro[isobenzofuran-1(3H),9′-[9H]xanthene]-3-one (YAMADA ETAC—CAS No. 59129-79-2), 6′-(diethylamino)-2′-[[3-(trifluoromethyl)phenyl]amino]spiro[isobenzofuran-1(3H),9′-[9H]xanthene]-3-one (YAMADA BLACK 100—CAS No. 68134-61-2), 3,3-Bis[2-[4-(dimethylamino)phenyl]-2-(4-methoxyphenyl)vinyl]-4,5,6,7-tetrachlorophthalide (YAMADA NIR BLACK 78—CAS No. 113915-68-7), 7-[4-(diethylamino)-2-hexoxyphenyl]-7-(1-ethyl-2-methylindol-3-yl)furo[3,4-b]pyridin-5-one (YAMADA BLUE 220—CAS No. 114090-18-5), 3-(4-Diethylamino-2-hexyloxyphenyl)-3-(2-methyl-1-ethylindol-3-yl)-4-azaphthalide (YAMADA BLUE 203—CAS No. 98660-18-5), 3,3-Bis(2-methyl-1-octyl-1H-indol-3-yl)isobenzofuran-1(3H)-one (PERGASCRIPT RED 16—CAS No. 50292-95-0).

In embodiments, when a leuco dye is used, it is preferably partially or completely dissolved in a solvent such as dibutyl adipate before being incorporated in a transparent varnish such as overprint offset varnish. The coating can also contain a void former, such as, for example, Celite, which has a specific geometry that forms a void cell in the coating. The void cell functions by capillary action. Particularly, the Celite is used to hold the developed color former in a cell to prevent the dye penetration into and across the substrate in order to prevent smearing of the developed dye.

In accordance with the disclosed subject matter, a color developer can react with a Leuco dye to break open the lactone ring include phenols or aromatic amines, other colorants, hydrogen bonding agents, Bronsted acids like carboxylic acids or metal salts or Lewis acids, and mixtures thereof. Specific developers that can be used include gallic acid (CAS No. 149-91-7), propyl gallate (CAS No. 121-79-9), butyl gallate (CAS No. 1083-41-6), decyl gallate (CAS No. 19198-75-5), octyl gallate (CAS No. 1034-01-1), dodecyl (or lauryl) gallate (CAS No. 1166-52-5), Zinc 3,5-bis(alpha-methylbenzyl) salicylate (CAS No. 53770-52-8), Zinc Salicylate (CAS No. 16283-53-0), salicylic acid (CAS No. 69-72-7) and its salts and esters, 3,5-Di-tert-butylsalicylic acid (abbreviated TBSA with CAS No. 19715-19-6), Benzyl 4-hydroxybenzoate (abbreviated PHBB with CAS No. 94-18-8), 4,4′-Dihydroxybenzophenone (CAS No. 611-99-4), 2,4′-dihydrohybenzophenone (CAS No. 131-56-6), 2,2′,4,4′-Tetrahydroxybenzophenone (CAS No. 131-55-5), 2,2-bis(phydroxyphenyl) propane (common name of Bisphenol A with a CAS No. 80-05-7), Bis(4-hydroxyphenyl)methane (common name of Bisphenol F with a CAS No. 620-92-8), 4-Hydroxyphenyl sulfone (common name of Bisphenol S with a CAS No. 80-09-1), bis-(3-allyl-4-hydroxyphenyl) sulfone (CAS No. 41481-66-7), 4-[4′-[(1′-methylethyloxy) phenyl]sulfonyl]phenol (CAS No. 191680-38-8), 4-hydroxyphenyl 4-isoprooxyphenylsulfone (also known as D-8 with a CAS No. 95235-30-6), Phenol, 4-[[4-(2-propen-1-yloxy)phenyl]sulfonyl] (abbreviated BPS-MAE with a CAS No. 7042-18-7), 4-Hydroxy-4′-benzyloxydiphenylsulfone (abbreviated BPS-MPE with a CAS No. of 63134-33-8), Urea Urethane Compound with a CAS No. 321860-75-7, 4,4′-bis(N-carbamoyl-4-methylbenzenesulfonamide)diphenylmethane (abbreviated BTUM with a CAS No. 151882-81-4), 2,4′-Bis(hydroxyphenyl)sulfone (CAS No. 5397-34-2), 4,4′-(1-Phenylethylidene)bisphenol (also known as Bisphenol AP with a CAS No. 1571-75-1), 2,2′-Bis(4-hydroxy-3-methylphenyl)propane (also known as Bisphenol C with a CAS No. 79-97-0), Methyl bis(4-hydroxyphenyl)acetate (CAS No. 5129-00-0), 4,4′-Isopropyllidenebis(2-phenylpheno) (known as BisOPP-A with a CAS No. 24038-68-4), 1,7-bis(4-hydroxyphenylthio)-3,5-dioxaheptane (CAS No. 93589-69-6), 2,2-Bis(4-hydroxyphenyl)butane (abbreviated Bisphenol B with CAS No. 77-40-7), p-Dihydroxylbenzene (also known as hydroxyquinone with CAS No. 123-31-9), p-Methylphenol (also known as p-cresol with CAS No. 106-44-5), m-Nitrobenzoic acid(CAS No. 121-92-6), m-Aminobenzoic acid (CAS No. 99-05-8), tannic acid (CAS No. 1401-55-4), 1,2,3-triazoles (CAS No. 288-36-8), thioureas, calcium chloride (CAS No. 10043-52-4), magnesium chloride decahydrate, phenolphthalein, cresol red, N-(p-Toluenesulfonyl)-N′-(3-p-toluenesulfonyloxyphenyl)urea (known as PERGAFAST 201 with a CAS No. 232938-43-1), Phenol, 4,4′-sulfonylbis-, polymer with 1,1′-oxybis[2-chloroethane] (known as D-90 with a CAS No. 191680-83-8), MBHA (CAS No. 5129-00-0), aluminum trichloride, aluminum distearate monochloride, 4,4′-Dihydroxybenzophenone (CAS No. 611-99-4), Benzyl 4-Hydroxybenzoate (CAS No. 94-18-8).

Others examples of suitable developers include zinc chloride, phenol resins, 4-tert-butylphenol, (x-naphthol, [3-naphthol, 4-acetylphenol, 4-tert-octylphenol, 4,4′-sec-butylidenephenol, 4-phenylphenol, 4,4′-dihydroxydiphenylmethane, 4,4′-isopropylidene diphenol, hydroquinone, 4,4′cyclohexylidene diphenol, 4,4-dihydroxy diphenyl sulfide, 4,4′-thiobis(6-tert-butyl-3-methylphenol), 4,4′-dihydroxydiphenyl sulfone, hydroquinone monobenzyl ether, 4-hydroxybenzophenone, 2,4-dihydroxybenzophenone, 2,4,4′trihydroxybenzophenone, 2,2′,4,4′tetrahydroxybenzophenone, dimethyl 4-hydroxyphthalate, methyl 4-hydroxybenzoate, ethyl 4-hydroxybenzoate, propyl 4-hydroxybenzoate, sec-butyl 4-hydroxybenzoate, pentyl 4-hydroxybenzoate, phenyl 4-hydroxybenzoate, benzyl 4-hydroxybenzoate, tolyl 4-hydroxybenzoate, chlorophenyl 4-hydroxybenzoate, phenylpropyl 4-hydroxybenzoate, phenyl ethyl 4-hydroxybenzoate, p-chlorobenzyl 4-hydroxybenzoate, p-methoxybenzyl 4-hydroxybenzoate, benzoic acid and its salts, other benzoic acid esters and their salts, phenol polymers and like phenol compounds.

Further examples of suitable developers also include inorganic compounds like silica and natural or synthetic silicates like clays, zeolites and the like, as well as inorganic and organic compounds of polyvalent metals like oxides, halides, carbonates, sulfates, nitrates, acetates, formates, oxalates, benzoates, acetylacetonates, stearates, salicylates, hydroxides like sodium hydroxide and potassium hydroxide, and hydroxides of magnesium, aluminum, calcium, titanium, zinc, cadmium, nickel, cobalt, iron, manganese, copper, vanadium, magnesium chloride, calcium chloride, zinc chloride, zinc bromide, zinc iodide, zinc oxide, zinc stearate, zinc glycinate, zinc resinates, aluminum trichloride, aluminum oxide, aluminum stearate, aluminum glycinate, aluminum acetylacetonate etc., silica and silicates as well as compounds of zinc, aluminum, copper, and magnesium are preferred in embodiments. Among metallic compounds, zinc compounds are the most preferred in certain embodiments.

In some embodiments where the color developer is dissolved in a fragrance, zinc chloride is preferably used as the color developer.

Another example of a suitable color reaction involves the reaction between an halochromic compound and an acid or a base. Examples of suitable halochromic compounds are Cresolphthalein (meta), Cresol Purple, Thymol Blue, Methyl Orange—Xylene Cyanol, Bromophenol Blue, Congo Red, Methyl Orange, Alizarin Red S, Bromocresol Green, Dichlorofluorescein, Methyl Red, Bromocresol Green, Bromocresol Purple, Chlorophenol Red, Bromothymol Blue, Phenol Red, Naphtholphtalein (alpha), Phenolphthalein, Cresolphthalein (ortho), Thymolthalein and Indigo Carmine.

Examples of suitable acids include organic acid such as citric acid, lactic acid, tartaric acid and malic acid, as well as inorganic acids like hydrochloric acid and phosphoric acid.

Examples of suitable bases include organic acid salts, sodium hydroxide, potassium hydroxide and carbonate.

In the case of a color change with halochromic compounds, in embodiments, the fragrance is preferably in solution in water in the case of a water-soluble fragrance or is emulsified in water in the case of an oil-soluble fragrance.

The scented color-changing system can also comprise one or more additives such as brightness pigment, surfactant, binder, antioxidants, buffers, desensitizer, spacers, solvent and cosolvent, binder and void cells.

Reference will now be made in detail to the various exemplary embodiments of the disclosed subject matter, which are illustrated in the accompanying drawings. The structure and corresponding methods of operation of the disclosed subject matter will be described in conjunction with this detailed description.

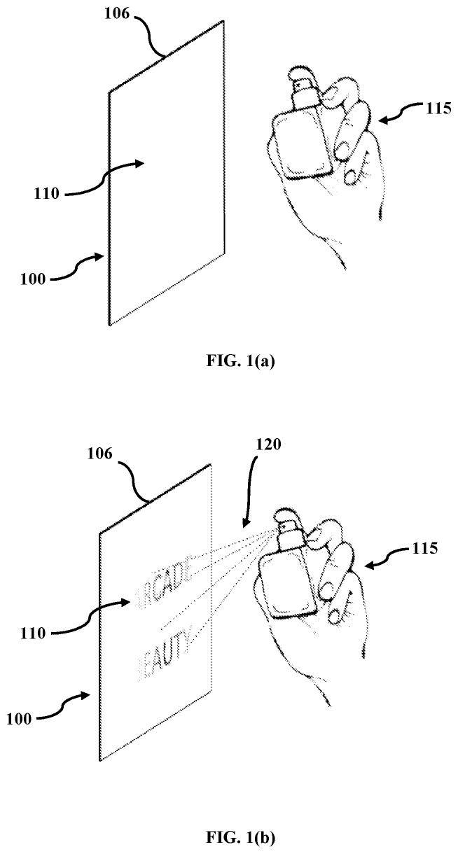

The accompanying figures, where like reference numerals refer to identical or functionally similar elements throughout the separate views, serve to further illustrate various embodiments and explain various principles and advantages all in accordance with the disclosed subject matter. For purpose of explanation and illustration, and not limitation, exemplary embodiments of the scented color-changing system in accordance with the disclosed subject matter are shown in FIGS. 1-6.

For purpose of illustration, and not limitation, reference is now made to FIGS. 1(a) & (b), which show a spray embodiment of the scented color-changing system 100. In this embodiment, the scented color-changing system 100 includes a substrate 106, a color message 110 made from a color former disposed on the substrate 106, a user 115, and a fragrance 120 containing a color developer. FIG. 1(a) shows the scented color-changing system 100 prior to the spraying of the fragrance 120 by the user 115. As shown, the color message 110 is not yet visible.

In operation, as shown in FIG. 1(b), the user 115 sprays fragrance 120 on the substrate and a color message appears. The paper is printed in the form of a message 110 with an invisible coating containing the color former (e.g., leuco dye). The color developer (e.g., zinc chloride) is solubilized in the fragrance 120 with ethanol and is sprayed on the paper. After spraying, the substrate 106 absorbs some of the fragrance 120, thereby combining some of the color former and color developer. The two reagents react together on the paper, which leads to a change of color of the invisible ink, revealing the message 110, as shown in FIG. 1(b). As the reagents react, the message 110 “fades in,” as shown.

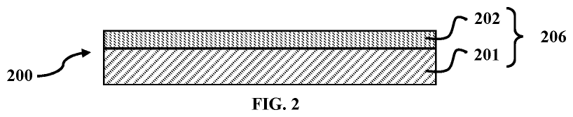

FIG. 2 shows an exemplary substrate structure of the scented color-changing system 200, corresponding to the embodiment described in FIG. 1. In this embodiment, a base layer (e.g., paper) 201 is printed with a transparent ink 202 which contains the color former to form substrate 206. In this embodiment, FIG. 2 shows the base layer 201 and ink 202 as separate layers (i.e., the ink 202 disposed on the base layer 201), but those of skill in the art will appreciate that other arrangements can be used. For example, the base layer 201 can absorb some or all the ink 202 such that the layers overlap. In this embodiment, the coloration of the ink changes when a mixture fragrance/color developer is applied (i.e., sprayed) on the substrate 206.

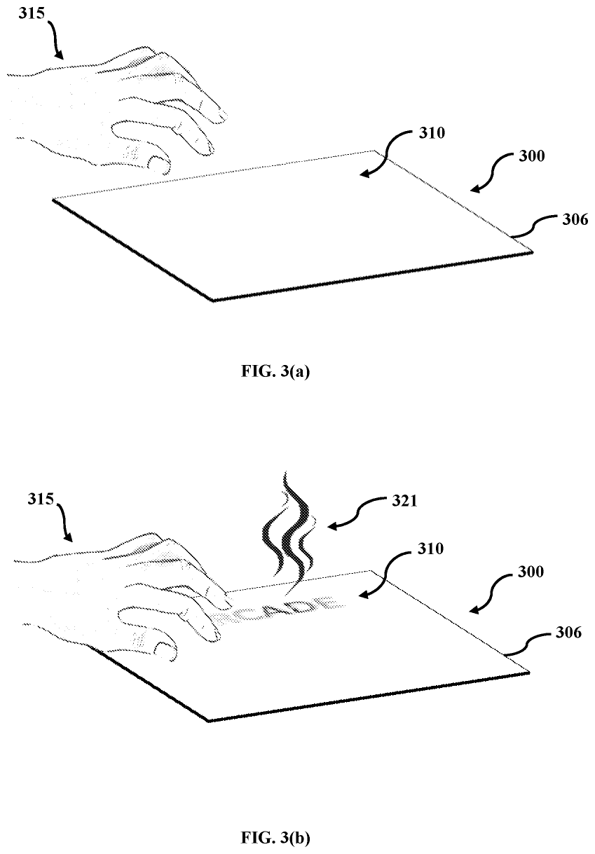

FIGS. 3(a) & (b) show a scratch-release embodiment of the scented color-changing system. In this embodiment, the scented color-changing system 300 includes a substrate 306, a color message 310 made from a color former disposed on the substrate 306, a user 315, and a fragrance 321 containing a color developer. FIG. 3(a) shows the scented color-changing system 300 prior to the release of the fragrance 321 by the user 315. As shown, the color message 310 is not yet visible.

In operation, as shown in FIG. 3(b), when the user 315 rubs or scratches the surface of the substrate 306, the fragrance 321 is released, and the message 310 appears. In this embodiment, an invisible ink containing a color former is applied on the substrate 306 in the form of the message 310. Capsules or microcapsules loaded with a mixture of fragrance 321 and a color developer are applied on or in the substrate 306. When the surface of the substrate 306 is abraded (e.g., rubbed or scratched), the capsules break and release the fragrance/developer mixture 321. The color former and developer react together leading to a coloration of the ink, causing the message 310 to appear. As the reagents react, the message 310 “fades in,” as shown.

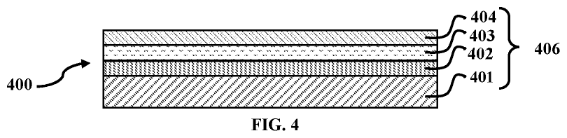

FIG. 4 shows an exemplary substrate structure of the scented color-changing system 400, corresponding to the embodiment described in FIG. 3. The substrate 406 is formed by a base layer (e.g., paper) 401, which is printed with a transparent ink 402, which contains the color former. These are akin to the layers shown in FIG. 2. In this embodiment, an intermediate layer 403 is also applied to avoid a preliminary color reaction between the two reagents before the application of the fragrance/color developer. Coating 404 is applied on top of the intermediate layer 403 and contains the fragrance/color developer microcapsules. The color reaction occurs when the intermediate layer 403 and microcapsules are broken such that the fragrance from the coating 404 mixes with the color former in the transparent ink 402.

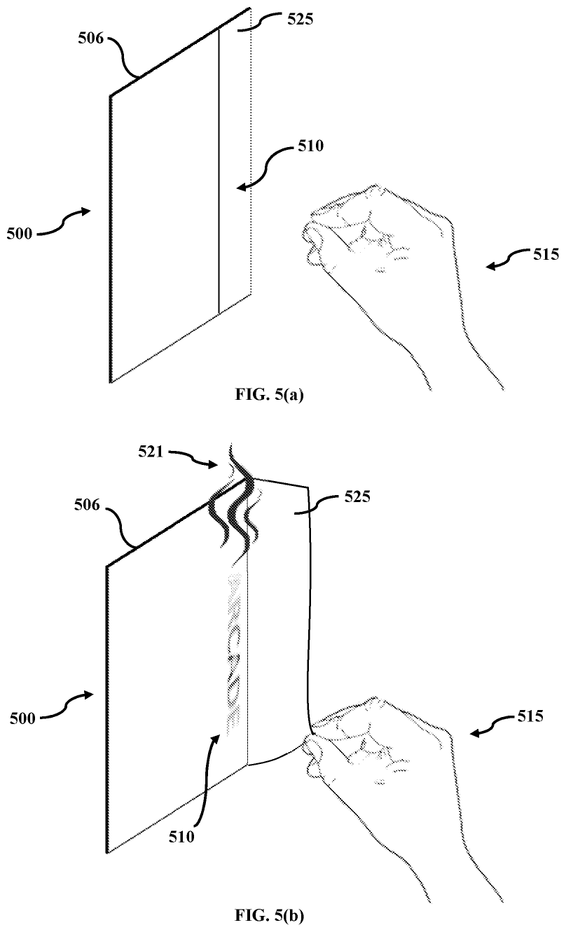

FIGS. 5(a) & (b) show a flap-release embodiment of the scented color-changing system. In this embodiment, the scented color-changing system 500 includes a substrate 506, a flap 525 that covers at least part of the substrate 506 when closed, a color message 510 made from a color former disposed on the substrate 506 and under the flap 525, a user 515, and a fragrance 521 containing a color developer. FIG. 5(a) shows the scented color-changing system 500 prior to the release of the fragrance by the user 415. The color message 510 is not yet visible.

In operation, as shown in FIG. 5(b), when the user 515 opens the flap 525, the fragrance 521 is released, and the message 510 appears. In this embodiment, an invisible ink containing a color former is applied on the substrate (under the flap 525) in the form of the message 510. Capsules or microcapsules loaded with a mixture of fragrance and a color developer are applied on or in the substrate 506 with a binder or adhesive to stick to the flap 525. When the flap 525 is opened, the capsules break and release the fragrance/developer mixture 521. The color former and developer react together leading to a coloration of the ink, causing the message 510 to appear. As the reagents react, the message 510 “fades in,” as shown.

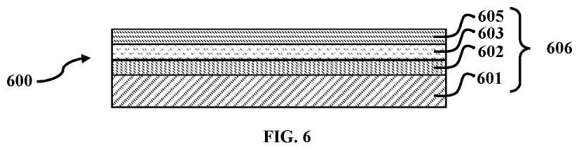

FIG. 6 shows an exemplary substrate structure of the scented color-changing system 600, corresponding to the embodiment described in FIG. 5. The substrate 606 is formed by a base layer (e.g., paper) 601, which is printed with a transparent ink 602, which contains the color former. In this embodiment, an intermediate layer 603 is also applied to avoid a preliminary color reaction between the two reagents before the release of the fragrance/color developer. These layers are akin to the layers shown in FIG. 4. Microcapsules 605 are applied on top of the intermediate layer 603 and contain the fragrance/color developer mixture. The color reaction occurs when the intermediate layer 603 and the microcapsules 605 are broken such that the fragrance/developer mixes with the color former in the transparent ink 602.

Exemplary embodiments of the scented color-changing system in accordance with the present disclosure will now be described.

### Example 1: Color Change by Fragrance Spraying

Tables 1 and 2 list the ingredients and composition of a scented color-changing system in accordance with the present disclosure.

TABLE 1
Color Former Coating Composition

| Component | Weight % |
|---|---|
| Oil-based varnish KB-3790 | 67 |
| Dibutyl adipate | 9 |
| CVL-K | 1 |
| Cellite 281 | 20 |
| Titanium dioxide | 3 |

TABLE 2
Color Developer Perfume Solution Composition

| Component | Weight % |
|---|---|
| Commercial alcoholic | 85 |
| Perfume solution | |
| Zinc chloride | 15 |

In Example 1, the Leuco dye CKL-K is dissolved at 50° C. in dibutyl adipate. Then the Leuco dye solution is absorbed on the Celite 281. The wet Celite and the titanium dioxide are added and mixed with the varnish (Table 1).

The Leuco dye varnish is printed on an uncoated paper (16 pts) by sheetfed offset printing process to make an invisible message, which will appear when the modified perfume is sprayed on the paper. When the color developer perfume solution (Table 2) is sprayed on the treated paper, the brand name of the fragrance (or other suitable message) appears in blue.

### Example 2: Color Change by Capsule Rubbing

Tables 3 and 4 list the ingredients and composition of a scented color-changing system in accordance with the present disclosure.

TABLE 3
Color Former Fragrance Solution Composition

| Component | Weight % |
|---|---|
| Leuco dye Red 520 | 1.8 |
| Leuco dye CVL-K | 0.8 |
| Fragrance oil | 97.1 |
| Butylated hydroxytoluene | 0.3 |

TABLE 4
Color Developer Coating

| Component | Weight % |
|---|---|
| FULACOLOR SR | 30 |
| DI Water | 64 |
| Polyvinyl alcohol | 6 |

In Example 2, two leuco dyes are dissolved in a fragrance oil at 40° C. (Table 3). Then, that solution is encapsulated by complex coacervation to get gelatin-gum Arabic capsules. The capsules are filtered and washed three times. Then a solution of polyvinyl alcohol and some spherical aliphatic polyurethane beads MicroTouch 800F are added to the capsules as binder and spacers respectively.

A color developer coating is prepared by mixing reactive acid clay (FULACOLOR SR) with water and polyvinyl alcohol (Table 4). The color developer coating is applied by flexography in the form of a message on a paper and the coating is dried. Then, the capsule slurry is applied on the top of color developer coating by flexography (rectangle shape) and dried.

The fragrance is released, and a purple message appears, when the surface of the paper is rubbed.

The foregoing description of exemplary embodiments, including examples, is presented only to describe, explain, and illustrate the broad concepts of the disclosed subject matter, and is not intended and should not be construed to limit the scope of the of the present disclosure. Various modifications and improvements may be made by those skilled in the art without departing from the scope. Thus, the disclosed subject matter includes all modifications and improvements that are within the scope of the following claims and their equivalents.

What is claimed is:

1. A scented color-changing system, comprising:
a substrate having a color-changing portion on which an ink mixture has been applied, the ink mixture containing a color former and having an initial color; and
a fragrance mixture containing volatile fragrance molecules and a color developer capable of chemically reacting with the color former to change the initial color to a changed color;
wherein application of at least some of the fragrance mixture to the color-changing portion of the substrate causes the initial color to change to the changed color in response to the chemical reaction between the color former and color developer.

2. The scented color-changing system of claim 1, wherein the substrate comprises an absorbent support.

3. The scented color-changing system of claim 2, wherein the absorbent support is paper.

4. The scented color-changing system of claim 1, wherein the color former is a leuco dye.

5. The scented color-changing system of claim 2, wherein the color developer is zinc chloride.

6. The scented color-changing system of claim 1, wherein the color developer is a halochromic compound and the color developer is an acid or a base.

7. The scented color-changing system of claim 1, wherein the application of at least some of the fragrance mixture to the color-changing portion of the substrate comprises spraying the fragrance mixture on the substrate.

8. The scented color-changing system of claim 1, wherein the fragrance mixture is encapsulated in microcapsules disposed in a coating on the substrate.

9. The scented color-changing system of claim 8, wherein the application of at least some of the fragrance mixture to the color-changing portion of the substrate comprises rupturing some of the microcapsules to release some of the fragrance mixture.

10. The scented color-changing system of claim 9, wherein the rupturing comprises abrading the color-changing portion of the substrate.

11. The scented color-changing system of claim 9, further comprising:
a flap disposed on the substrate and covering the color-changing portion; and
a peelable adhesive disposed between the substate and the flap,
wherein the rupturing comprises peeling the flap at least partly away from the substrate.

12. The scented color-changing system of claim 8, further comprising a barrier layer disposed between the substrate and the coating that chemically separates the color former and color developer, wherein the application of at least some of the fragrance mixture to the color-changing portion of the substrate further comprises rupturing the barrier layer.
